(19) United States
(12) Patent Application Publication
(10) Pub. No.: US 2023/0272129 A1
(43) Pub. Date: Aug. 31, 2023

(54) POLYMER OF 2-ACRYLAMIDO-2-METHYLPROPANE SULFONIC ACID OR THE SALTS THEREOF

(71) Applicant: SNF Group, Andrezieux Boutheon (FR)

(72) Inventors: Cédrick FAVERO, Andrezieux Boutheon (FR); Johann KIEFFER, Andrezieux Boutheon (FR); Benoît LEGRAS, Andrezieux Boutheon (FR)

(73) Assignee: SNF Group, Andrezieux Boutheon (FR)

(21) Appl. No.: 18/044,926

(22) PCT Filed: Aug. 19, 2021
(86) PCT No.: PCT/FR2021/051475

§ 371 Date: Mar. 10, 2023

(30) Foreign Application Priority Data
Sep. 11, 2020 (FR) 2009216

(51) Int. Cl. C08F 20/58

(52) U.S. Cl. CPC C08F 20/58

(57) ABSTRACT

The present invention relates to a polymer obtained at least from a quantity A of 2-acrylamido-2-methylpropane sulfonic acid, in acid and/or salified form, characterized in that the quantity A of 2-acrylamido-2-methylpropane sulfonic acid contains 250 to 20,000 ppm by weight of 2-methyl-2-propenyl-sulfonic acid, in acid and/or salified form. The present invention also relates to the use of this polymer as a flocculant, viscosity reducing agent, thickener, absorbent, friction reducing agent or plasticizer or superplasticizer, in particular in a field chosen from among oil and gas recovery, water treatment, sludge treatment, paper manufacture, construction, mining, cosmetic product formulation, detergent formulation, textile manufacture and agriculture.

### FIELD OF THE INVENTION

This invention relates to a polymer of 2-acrylamido-2-methylpropane sulfonic acid or at least one of its salts. More specifically, the invention relates to a polymer obtained from the 2-acrylamido-2-methylpropane sulfonic acid monomer or at least one of its salts, said monomer containing impurities.

### PRIOR ART

2-Acrylamido-2-methylpropane sulfonic acid (ATBS) and its salts are widely used as a raw material to obtain polymers used as a dispersant, thickener, flocculant, or superabsorbent in various sectors such as the oil, construction, textiles, water treatment (desalination of sea water, mineral industry, etc.), or cosmetics industries.

The reaction implemented in the process for the synthesis of 2-acrylamido-2-methylpropane sulfonic acid corresponds to the reaction scheme below, in which the acrylonitrile is present in excess so as to be both the solvent of the reaction and the reactant. Acrylonitrile is brought into contact with fuming sulfuric acid (oleum) and isobutylene.

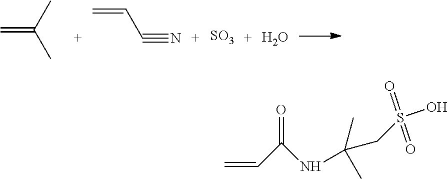

2-Acrylamido-2-methylpropane sulfonic acid (ATBS) is not soluble in acrylonitrile solvent. Therefore, the reaction product is in the form of a suspension of crystals in the reaction solvent.

The 2-acrylamido-2-methylpropane sulfonic acid is subsequently separated from the acrylonitrile, usually by filtration, and then dried. Drying the acrylamido-2-methyl-2-propane sulfonic acid is necessary in order to reduce the quantity of acrylonitrile and acrylamide that remains present in the crystal.

Subsequently, the 2-acrylamido-2-methylpropane sulfonic acid is purified in order to eliminate the impurities contained in the monomer. There are many purification techniques that may reduce the presence of these impurities. This purification thus makes it possible to avoid contamination of the monomer which, according to current knowledge, is detrimental to the correct polymerization of the monomer.

There are many methods for purifying 2-acrylamido-2-methylpropane sulfonic acid. Most often, 2-acrylamido-2-methylpropane sulfonic acid is redissolved in a hot solvent, in order to obtain a saturated solution. High purity crystals are obtained during the cooling process. Over time, different solvents have been used to improve this purification. Mention may be made of acetic acid, C1-C4 alcohols, ketones, or propionic acid.

Document US 2010/0274048 identifies two types of impurities present in 2-acrylamido-2-methylpropane sulfonic acid, 2-methyl-2-propenyl-sulfonic acid (IBSA) and 2-methylidene-1, 3-propylenedisulfonic acid (IBDSA). According to the authors, these two impurities act as chain transfer agents and strongly affect the polymerization when they are present above a certain concentration in the 2-acrylamido-2-methylpropane sulfonic acid monomer.

To solve this problem, the document describes a process for the manufacture of 2-acrylamido-2-methylpropane sulfonic acid in which the reaction conditions are optimized so that the concentration of sulfur trioxide is reduced, which has the effect of reducing the level of these impurities below 100 ppm, preferably below 30 ppm.

According to the authors, unpurified 2-acrylamido-2-methylpropane sulfonic acid, that consequently contains high levels of impurities, cannot be used as is to obtain 2-acrylamido-2-methylpropane sulfonic acid polymers with high molecular weight.

Documents JP 2010-270169, JP 2010-270170, and JP 2010-270168 describe polymers obtained from ATBS containing up to 120 ppm of IBSA. The comparative examples of these documents show that the presence of 200 ppm of IBSA is harmful.

It would therefore have been expected that the polymerization of 2-acrylamido-2-methylpropane sulfonic acid, which was not purified or which contained a high level of impurities, would lead to polymers having degraded properties. However, against all expectations, the Applicant discovered that it was possible to use 2-acrylamido-2-methylpropane sulfonic acid containing a substantial level of impurities and obtain high molecular weight polymers without significantly affecting their properties.

### DISCLOSURE OF THE INVENTION

This invention relates to a polymer obtained at least from a quantity A of 2-acrylamido-2-methylpropane sulfonic acid, in acid and/or salified form. Quantity A of 2-acrylamido-2-methylpropane sulfonic acid contains from 250 to 20,000 ppm by weight of 2-methyl-2-propenyl-sulfonic acid, in acid and/or salified form, advantageously 300 to 20,000 ppm.

The invention also relates to the use of said polymer in a field chosen from oil and gas recovery, water treatment, sludge treatment, papermaking, construction, the mining industry, cosmetics formulation, detergent formulation, textile manufacturing, and agriculture. Another subject-matter of the invention is the use of said polymer as a flocculant, viscosity-reducing agent, thickening agent, absorbing agent, or friction-reducing agent.

The polymer according to the invention is preferably a water-soluble polymer or a superabsorbent polymer.

Unless otherwise indicated, ppm and percentages are by weight.

By definition, a water-soluble polymer is a polymer which gives an aqueous solution when dissolved under stirring and with a concentration of 10 g/L in water at 25° C. The level of impurity of 2-methyl-2-propenyl-sulfonic acid and/or its salts (IBSA) is expressed in ppm by weight in the monomer of 2-acrylamido-2-methylpropane sulfonic acid and/or of its salts. In general, unless otherwise indicated, the term, “2-acrylamido-2-methylpropane sulfonic acid” or ATBS denotes the acid and/or the salified form. The same is true with 2-methyl-2-propenyl-sulfonic acid (IBSA) and for 2-methylidene-1,3-propylenedisulfonic acid (IBDSA). The acid form of an acid corresponds to the form of the Brønsted acid while the salified form (or a salt) corresponds to the Brønsted base of the acid, the counter-ion being advantageously chosen from among the alkali metals, alkaline earth metals, and ammoniums (primary, secondary, tertiary or quaternary).

According to the invention, it is possible to obtain high molecular weight polymers from 2-acrylamido-2-methylpropane sulfonic acid monomer containing a high level of IBSA impurity without the performance of the polymers being significantly and negatively impacted.

According to one preferred embodiment, the polymer is obtained from a quantity A of 2-acrylamido-2-methylpropane sulfonic acid, in acid and/or salified form, containing from 300 to 10,000 ppm by weight of 2-methyl-2-propenyl-sulfonic, in acid and/or salified form, more particularly 350 to 5000 ppm, even more particularly 400 to 2000 ppm, and even more particularly 500 to 1500 ppm.

The 2-acrylamido-2-methylpropane sulfonic acid (acid and/or salified form), used to prepare the polymer, may be the product of a process to manufacture 2-acrylamido-2-methylpropane sulfonic acid, after which it is not substantially purified. Preferably, it is not purified. It may also be a residue or waste or purge from a 2-acrylamido-2-methylpropane sulfonic acid purification process. In general, the purification process is advantageously carried out by a recrystallization process from a solvent such as acrylonitrile, acetic acid, or methanol. It may also be the purge, or the filtrate obtained during the ATBS production process, for example, according to the process described in document WO 2018/172676. The residue, waste, purge, or filtrate contains a variable quantity of ATBS. This may be quantity A of ATBS, or quantity B of ATBS (see below), or the sum of quantities A and B, of ATBS or the total quantity of ATBS used to prepare the polymer.

Thus, according to the invention, it is possible to polymerize a 2-acrylamido-2-methylpropane sulfonic acid monomer considered to be of low quality because it contains a high level of impurities and to obtain a high molecular weight polymer without the polymer characteristics being substantially affected. In other words, where it seemed necessary to purify 2-acrylamido-2-methylpropane sulfonic acid, this invention demonstrates that it is possible to reduce the requirement level of a purification step, ideally, dispense with the purification step of 2-acrylamido-2-methylpropane sulfonic acid, and obtain high molecular weight polymers with satisfactory performance.

According to one particular embodiment, quantity A of 2-acrylamido-2-methylpropane sulfonic acid, in its acid and/or salified form, also contains between 300 and 10,000 ppm by weight of 2-methylidene-1,3-propylenedisulfonic (IBDSA), in acid and/or salified form, more particularly between 350 and 5000 ppm, even more particularly between 400 and 2000 ppm, even more particularly between 500 and 1500 ppm.

Quite surprisingly, the Applicant noticed that it was thus possible to obtain high molecular weight polymers, even in the presence of a substantial quantity of IBSA and, optionally, of IBDSA.

When the polymer is a water-soluble polymer, it has a weight-average molecular weight advantageously greater than 100,000 g/mol, more advantageously greater than 500,000 g/mol, preferably greater than 1 million g/mol, more preferably greater than 1.5 million, even more preferably greater than 2 million, even more preferably greater than 3 million, even more preferably between 5 and 40 million, even more preferably between 7 and 30 million, even more preferably between 9 and 30 million, even more preferably between 10 and 25 million, even more preferably greater than 12 million.

By definition, superabsorbent polymers have an infinite molecular weight since they form a three-dimensional network. A superabsorbent is a cross-linked polymer capable, when it is in the form of a solid or gelled particle, of absorbing a large quantity of water, generally at least once its weight. They are obtained with the same quantities of ATBS as the water-soluble polymers.

The weight-average molecular weight of the polymer according to the invention is advantageously determined by the intrinsic viscosity of the polymer. The intrinsic viscosity may be measured by methods known to a person skilled in the art and may advantageously be calculated from the values of reduced viscosity for different concentrations of polymer by a graphical method consisting of noting the values of reduced viscosity (y-axis) with respect to the concentration (x-axis), and extrapolating the curve to zero concentration. The intrinsic viscosity value is plotted on the y-axis or using the least squares method. The molecular weight may then be determined by the Mark-Houwink equation:

[η]=KM^{α}

wherein:
[η] represents the intrinsic viscosity of the polymer determined by the solution viscosity measurement method,
K represents an empirical constant,
M represents the molecular weight of the polymer,
α represents the Mark-Houwink coefficient,
K and α depend on the particular polymer-solvent system.

The polymer according to the invention may be a homopolymer of 2-acrylamido-2-methylpropanesulfonic acid, or a copolymer additionally comprising at least one monomer chosen from the group comprising nonionic monomers, anionic monomers, cationic monomers, zwitterionic monomers, and mixtures thereof.

The polymer according to the invention is preferably obtained from a total quantity of 2-acrylamido-2-methylpropane sulfonic acid (acid and/or salified form) representing, relative to the total quantity of monomers, between 1 and 100% molar, preferably more than 10% molar, more preferably more than 20% molar, even more preferably more than 30% molar. This quantity may be greater than 40% molar, for example more than 50% molar, or more than 60% molar, or more than 70% molar, or more than 80% molar, or even more than 90% molar.

By definition, quantity A of ATBS corresponds to the quantity of ATBS used to prepare the polymer of the invention and having an IBSA concentration of between 250 and 20,000 ppm by weight, in other words greater than or equal to 250 ppm and less than or equal to 20,000 ppm. Quantity B of ATBS corresponds to the quantity of ATBS used to prepare the polymer of the invention and having an IBSA concentration strictly less than 300 ppm, preferably strictly less than 250 ppm, even more preferably strictly less than 200 ppm in weight. The total quantity of ATBS is the sum of quantities A and B and of the quantity of ATBS containing a concentration of IBSA strictly greater than 20,000 ppm of IBSA. In a preferred mode, the polymer is not obtained with a quantity of ATBS containing a concentration of IBSA strictly greater than 20,000 ppm of IBSA.

In a particularly preferred mode, the polymer is obtained from a quantity A of 2-acrylamido-2-methylpropane sulfonic acid (acid and/or salified form) representing, relative to the total quantity of ATBS, between 1 and 100% molar, preferably more than 2% molar, more preferably more than 5% molar, even more preferably more than 10% molar, even more preferably more than 20% molar, even more preferably more than 25% molar, even more preferably more than 30% molar, even more more than 40% molar, even more preferably more than 50% molar, even more preferably more than 60% molar, even more preferably more than 70% molar, even more preferably more than 80% molar, even more preferably more than 90% molar.

Thus, the polymer may be obtained at least from quantity A of ATBS (acid and/or salified form) and quantity B of ATBS (acid and/or salified form), quantity B containing strictly less than 300 ppm, preferably strictly less than 250 ppm, even more preferably strictly less than 200 ppm by weight of 2-methyl-2-propenylsulfonic acid (IBSA), in acid and/or salified form.

The polymer may be obtained at least from 2-acrylamido-2-methylpropane sulfonic acid, in acid and/or salified form, the total quantity of which contains strictly less than 20,000 ppm by weight of 2-methyl-2 acid.-propenyl-sulfonic, in acid and/or salified form.

In another particularly preferred embodiment concerning water-soluble polymers, the proportion D of quantity A relative to the total quantity of 2-acrylamido-2-methylpropane sulfonic acid, in acid and/or salified form, is determined from the ratio R defined according to the following equation (1):

$$R = \frac{{10}^{14}}{{[ IBSA ]}^{2} * {C}^{2} Mw} \qquad ( 1 )$$

wherein,
[IBSA] is the concentration, in ppm by weight, of 2-methyl-2-propenyl-1-sulfonic acid in quantity A;
Mw is the weight-average molecular weight of the polymer expressed in g/mol;

It is a coefficient equal to 0.2 when Mw is less than or equal to 1 million, equal to 0.6 when Mw is strictly greater than 1 million and less than or equal to 10 million, and equal to 0.8 when Mw is strictly greater than 10 million.

When R is strictly greater than 100, D is between 1 and 100%, preferably between 25 and 95%, more preferably between 50 and 90%.

When R is strictly greater than 50 and less than or equal to 100, D is between 1 and 90%, preferably between 25 and 85%, more preferably between 50 and 80%.

When R is strictly greater than 10 and less than or equal to 50, D is between 1 and 80%, preferably between 25 and 75%, more preferably between 50 and 70%.

When R is strictly greater than 5 and less than or equal to 10, D is between 1 and 60%, preferably between 2 and 50%, more preferably between 5 and 40%.

When R is strictly greater than 1 and less than or equal to 5, D is between 1 and 50%, preferably between 2 and 40%, more preferably between 4 and 30%.

When R is strictly greater than 0.1 and less than or equal to 1, D is between 1 and 30%, preferably between 2 and 25%, more preferably between 3 and 20%.

When R is strictly greater than 0.01 and less than or equal to 0.1, D is between 1 and 10%, preferably between 1 and 8%, more preferably between 1 and 6%.

When R is less than or equal to 0.01, D is between 1 and 5%, preferably between 1 and 4%, more preferably between 1 and 3%.

By way of example, if for an ATBS homopolymer, quantity A of ATBS is 80 mol % and quantity B of ATBS is 20 mol %, and this homopolymer is not obtained with a quantity of ATBS containing strictly more than 20,000 pm of IBSA, then the proportion D is equal to 80%. If for a copolymer of ATBS and acrylamide containing 50 mol % of acrylamide, quantity A of ATBS is 30 mol %, quantity B of ATBS is 20 mol % and this copolymer is not obtained with a quantity of ATBS containing strictly more than 20,000 ppm of IBSA, then the proportion D is equal to 60%.

The quantities of ATBS may be expressed in molar percentage or in percentage by weight. The ratios of these quantities are, by definition, unitless, for example D=A/(A+B) in the absence of ATBS containing strictly more than 20,000 pm of IBSA.

In a particular embodiment, the polymer may be obtained with quantity “a”, containing for example 280 ppm of IBSA and quantity “a′” containing for example 560 ppm of IBSA. Quantity A will be the sum of quantities “a” and “a” and the concentration of IBSA may easily be determined according to the proportion of the two quantities “a” and “a′”.

According to a preferred embodiment, the polymer of the invention is obtained from the salified form of 2-acrylamido-2-methylpropane sulfonic acid. The 2-acrylamido-2-methylpropane sulfonic acid is therefore preferably partially or totally salified before polymerization. The acid form of a monomer may be salified before and/or during and/or after the polymerization of the monomers.

The salified form is advantageously obtained from a compound chosen from an alkali or alkaline-earth metal hydroxide, an alkali or alkaline-earth metal oxide, ammonia, an amine of the following formula NR₁R₂R₃ (R₁, R₂ and R₃, identical or different, being advantageously hydrocarbon groups, in particular alkyls) and an alkali or alkaline-earth metal carbonate. A preferred alkali metal is sodium.

As previously explained, the polymer of the invention may be a copolymer obtained from 2-acrylamido-2-methylpropane sulfonic acid (acid and/or salified form) and at least one nonionic monomer, and/or at least one anionic monomer, and/or at least one cationic monomer and/or at least one zwitterionic monomer.

The nonionic monomer(s) may be chosen, in particular, from the group comprising water-soluble vinyl monomers. Preferred monomers belonging to this class are, for example, acrylamide, methacrylamide, N-isopropylacrylamide, N,N-dimethylacrylamide, N,N-diethylacrylamide and N-methylolacrylamide. N-vinylformamide (NVF), N-vinyl acetamide, N-vinylpyridine and N-vinylpyrrolidone (NVP), N-vinyl imidazole, N-vinyl succinimide, acryloyl morpholine (ACMO), acryloyl chloride, glycidyl methacrylate, glyceryl methacrylate, diacetone acrylamide, and isoprenol may also be used. A preferred nonionic monomer is acrylamide.

Anionic monomers may be selected from a wide group. These monomers may present vinyl functions, in particular acrylic, maleic, fumaric, malonic, itaconic, or allylic, and contain a carboxylate, phosphonate, phosphate, sulfate, sulfonate group, or another group with an anionic charge. The anionic monomer may be in acid form or else in salified form, for example an alkaline-earth metal salt, an alkali metal salt, or an ammonium salt. Examples of suitable monomers include acrylic acid; methacrylic acid; itaconic acid; crotonic acid; maleic acid; fumaric acid; acrylamide undecanoic acid; 3-acrylamido 3-methylbutanoic acid; maleic anhydride; strong acid type monomers having for example a sulfonic acid or phosphonic acid function such as vinylsulfonic acid, vinylphosphonic acid, allylsulfonic acid, methallylsulfonic acid, 2-sulfoethylmethacrylate, sulfopropylmethacrylate, sulfopropylacrylate, allylphosphonic acid, styrene sulfonic acid, 2-acrylamido-2-methylpropane disulfonic acid; and the water-soluble salts of these monomers such as their alkali metal, alkaline earth metal, or ammonium salts. In this list, the strong acid type monomers mentioned as having a sulfonic acid type function do not include quantity A of 2-acrylamido-2-methylpropane sulfonic acid. Alternatively, they include quantity B of purified 2-acrylamido-2-methylpropane sulfonic acid comprising strictly less than 300 ppm, preferably strictly less than 250 ppm, even more preferably strictly less than 200 ppm of IBSA. Preferably, they exclude any quantity of ATBS containing strictly more than 20,000 ppm by weight of IBSA.

The cationic monomers may be chosen, in particular, from monomers derived from vinyl type units, in particular acrylamide, acrylic, allylic or maleic, these monomers having a phosphonium or ammonium function. The ammonium is advantageously tertiary or quaternary, more advantageously quaternary. Particularly and not exclusively, quaternized dimethylaminoethyl acrylate (ADAME), quaternized dimethylaminoethyl methacrylate (MADAME), dimethyldiallylammonium chloride (DADMAC), acrylamido propyltrimethyl ammonium chloride (APTAC), and methacrylamido propyltrimethyl ammonium chloride (MAPTAC) may be cited.

The acidified salts are obtained by means known to a person skilled in the art, and in particular by protonation. The quaternized salts are also obtained by means known to those skilled in the art, in particular by reaction with benzyl chloride, methyl chloride (MeCl), aryl or alkyl chlorides, or dialkyl sulfates such as dimethyl sulfate. The quaternizing agent may be chosen from alkyl chlorides, dialkyl sulfates and alkyl halides. Preferably, the quaternizing agent is chosen from methyl chloride and diethyl sulfate.

Advantageously, the zwitterion monomer(s) that may be used within the framework of the invention may be derived from a unit of the vinyl type, in particular acrylamide, acrylic, allylic or maleic, this monomer having an amine or ammonium functional group (advantageously quaternary) and an acid functional group of the carboxylic (or carboxylate), sulfonic (or sulfonate) or phosphoric (or phosphate) type and mixtures thereof. Examples of zwitterion monomers are, e.g., dimethylaminoethyl acrylate derivatives, such as 2-((2-(acryloyloxy)ethyl)dimethylammonio)ethane-1-sulfonate, may comprise in particular but are not limited to, 3-((2-(acryloyloxy) ethyl) dimethylammonio) propane-1-sulfonate, 4-((2-(acryloyloxy) ethyl) dimethylammonio) butane-1-sulfonate, [2-(acryloyloxy) ethyl](dimethylammonio)acetate, dimethylaminoethyl methacrylate derivatives such as 2-((2-(methacryloyloxy)ethyl)dimethylammonio)ethane-1-sulfonate, (methacryloyloxy)ethyl)dimethylammonio)propane-(methacryloyloxy)ethyl)dimethylammonio)butane-1-sulfonate, 1-sulfonate, (methacryloyloxy)ethyl](dimethylammonio)acetate, propylacrylamide dimethylamino derivatives such as 2-((3-acrylamidopropyl)dimethylammonio)ethane-1-sulfonate, 3-(3-acrylamidopropyl)dimethylammonio)propane-1-sulfonate, 4-((3-acrylamidopropyl) dimethylammonio) butane-1-sulfonate, [3-(acryloyl) oxy) propyl] (dimethylammonio) acetate, dimethylamino propyl methylacrylamide, or derivatives such as 2-((3-methacrylamidopropyl) dimethylammonio) ethane-1-sulfonate, 3-((3-me dimethylammonio)propane-1-sulfonate 4-((3-methacrylamidopropyl)dimethylammonio)butane-1-sulfonate and propyl[3-(methacryloyloxy)](dimethylammonio)acetate.

The total quantity of nonionic, anionic, cationic or zwitterionic monomers of the preceding lists may be between 1 and 99% molar, generally between 10 and 80%, more generally between 20 and 50% molar. The person skilled in the art will know how to adjust the proportion of each monomer so that the sum of the monomers represents 100% molar, including ATBS. Unless otherwise indicated, the percentages are molar percentages expressed relative to the total number of all the monomers, including ATBS.

Monomers having a hydrophobic character may also be used in the preparation of the polymer of the invention. They are preferably chosen from the group consisting of esters of (meth)acrylic acid having an alkyl, arylalkyl, propoxylated, ethoxylated, or ethoxylated and propoxylated chain; (meth)acrylamide derivatives having an alkyl, arylalkyl propoxylated, ethoxylated, ethoxylated and propoxylated, or dialkyl chain; alkyl aryl sulfonates.

When a monomer having a hydrophobic character is used for the preparation of the water-soluble (co)polymer, its quantity is advantageously in the range between 0.001 and 3 mol %.

In one preferred mode, the polymers according to the invention do not contain acrylonitrile monomer or its derivatives, nor styrene monomer or its derivatives.

In a preferred embodiment, the polymer according to the invention may have a linear, branched, star (star-shaped), comb (comb-shaped), dendritic or block structure. This structure may be obtained, according to the general knowledge of the person skilled in the art, for example by selecting the initiator, the transfer agent, the polymerization technique such as controlled radical polymerization known as RAFT (reversible chain transfer by addition-fragmentation), NMP (Nitroxide Mediated Polymerization) or ATRP (radical polymerization by transfer of atoms, from the “Atom Transfer Radical Polymerization”), incorporation of structural monomers, or concentration.

According to the invention, the polymer is advantageously linear or structured. The term, structured polymer means a non-linear polymer which has side chains so as to obtain, when this polymer is dissolved in water, a strong state of entanglement leading to very high low-gradient viscosities.

The polymer may also be structured:
by at least one structural agent, which may in particular be chosen from the group comprising polyethylenically unsaturated monomers (having at least two unsaturated functions), such as for example vinyl, allylic, acrylics and, epoxy and mention may be made, for example, of methylene bis acrylamide (MBA), triallyamine, or tetraallylammonium chloride or 1,2 dihydroxyethylene bis-(N-acrylamide), and/or
by macroinitiators such as polyperoxides, polyazos and polytransfer agents such as polymercaptan (co)polymers, and polyols, and/or
by functionalized polysaccharides.

The quantity of structural agent, advantageously a branching/crosslinking agent, in the monomer mixture is advantageously less than 4% by weight relative to the quantity of monomer(s), more advantageously less than 1%, and even more preferably less than 0.5%. According to a particular embodiment, it may be at least equal to 0.00001% by weight relative to the quantity of monomer(s).

In general, the polymer does not require the development of a particular polymerization process. Indeed, it can be obtained by all the polymerization techniques well-known to a person skilled in the art. This may, in particular, include solution polymerization; gel polymerization; precipitation polymerization; emulsion polymerization (aqueous or reverse); suspension polymerization; reactive extrusion polymerization; water-in-water polymerization; or micellar polymerization.

Polymerization is generally a free radical polymerization, preferably by inverse emulsion polymerization or gel polymerization. Free radical polymerization includes free radical polymerization using UV, azo, redox or thermal initiators as well as controlled radical polymerization (CRP) and matrix polymerization techniques.

The invention also has as its subject-matter the use of the polymer according to the invention in oil and gas recovery, water treatment, sludge treatment, papermaking, construction, the mining industry, the formulation of cosmetic products, the formulation of detergents, textile manufacturing, or agriculture. The invention also has as its subject-matter the use of said polymer as a flocculant, thickening agent, absorbing agent or friction reducing agent. In the manufacture of paper, the polymer of the invention may advantageously be used to improve the dry strength of paper. In construction, the polymer of the invention may advantageously be used as a plasticizer or superplasticizer.

Another subject-matter of the invention is the use of the polymer according to the invention as a flocculant, viscosity-reducing agent, thickening agent, absorbing agent, friction-reducing agent, plasticizer or superplasticizer.

Quite surprisingly, it is thus possible to polymerize a 2-acrylamido-2-methylpropane sulfonic acid monomer considered to be of low quality (250-20,000 ppm, advantageously 300-20,000 ppm by weight of IBSA) and obtain a high molecular weight polymer without the characteristics of the polymer being substantially affected.

In other words, where it seemed necessary to purify 2-acrylamido-2-methylpropane sulfonic acid, the invention demonstrates that it is possible to reduce the requirement level for a purification step, and ideally to dispense with a purification step for 2-acrylamido-2-methylpropane sulfonic acid and obtain high molecular weight polymers of satisfactory performance.

The following examples are given only by way of illustration of the subject-matter of the invention, without limiting it in any way.

### EXAMPLES

### Example 1 (comparative): Preparation of a Homopolymer of 2-acrylamido-2-methylpropane sulfonic acid

To a 2000 mL beaker are added 390.5 g of deionized water, 262 g of 50% lye (by weight in water) and 847.5 g of 2-acrylamido-2-methylpropane sulfonic acid crystals containing 132 ppm IBSA.

The solution thus obtained is cooled to between 5 and 10° C. and transferred to an adiabatic polymerization reactor, nitrogen bubbling is carried out for 30 minutes in order to eliminate any trace of dissolved oxygen.

The following are then added to the reactor:
0.45 g of 2,2′-azobisisobutyronitrile,
1.5 mL of an aqueous solution at 2.5 g/L of 2,2′-Azobis[2-(2-imidazolin-2-yl)propane] dihydrochloride,
1.5 mL of an aqueous solution at 1 g/L of sodium hypophosphite,
1.5 mL of an aqueous solution at 1 g/L of tert-butyl hydroperoxide,
1.5 mL of an aqueous solution containing 1 g/L of ammonium sulfate and iron(II) hexahydrate (Mohr's salt).

After a few minutes, the nitrogen inlet is closed, and the reactor is closed. The polymerization reaction takes 4 hours to reach a peak temperature. The resulting rubbery gel is chopped and dried to obtain a coarse powder which is then ground and sieved to obtain the 2-acrylamido-2-methylpropane sulfonic acid homopolymer in powder form.

### Example 2 (comparative): Preparation of an acrylamide/2-acrylamido-2-methylpropane sulfonic acid copolymer (75/25 mol %)

549.55 g of deionized water, 520.5 g of acrylamide in 50% solution (by weight in water), 97.6 g of 50% sodium hydroxide solution (by weight in water) and 316.2 g of 2-acrylamido-2-methylpropane sulfonic acid crystals containing 132 ppm of IBSA are added a 2000 mL beaker. The same polymerization and gel processing procedures as in Example 1 are carried out. A polymer in powder form is obtained.

### Example 3 (comparative): Preparation of an acrylamide/2-acrylamido-2-methylpropane sulfonic acid copolymer (75/25 mol %)

The same preparation as in Example 2 is carried out, with the only difference being that 0.5 mL of a 1 g/L aqueous solution of sodium hypophosphite is used instead of 1.5 mL. A polymer in powder form is obtained.

### Example 4 (Invention)

The same preparation as in Example 1 is performed with the only difference being that 53% by weight of the 2-acrylamido-2-methylpropane sulfonic acid, or 449.175 g, are substituted with 2-acrylamido-2-methylpropane sulfonic acid containing 1042 ppm IBSA. Proportion D is 53%.

### Example 5 (Invention)

The same preparation as in Example 2 is performed with the only difference being that 55% of the 2-acrylamido-2-methylpropane sulfonic acid, or 173.91 g, are substituted with 2-acrylamido-2-methylpropane sulfonic acid containing 715 ppm IBSA. Proportion D is 55%.

### Example 6 (Invention)

The same preparation as in Example 3 is performed with the only difference being that 23% of the 2-acrylamido-2-methylpropane sulfonic acid, or 72.726 g, are substituted with 2-acrylamido-2-methylpropane sulfonic acid containing 1210 ppm IBSA. Proportion D is 23%.

### Example 7 (Invention)

The same preparation as in Example 1 is performed with the only difference being that 58% by weight of the 2-acrylamido-2-methylpropane sulfonic acid, or 491.55 g, are substituted with 2-acrylamido-2-methylpropane sulfonic acid containing 1042 ppm IBSA. Proportion D is 58%.

### Example 8 (Invention)

The same preparation as in Example 2 is performed with the only difference being that 60% of the 2-acrylamido-2-methylpropane sulfonic acid, or 189.72 g, are substituted with 2-acrylamido-2-methylpropane sulfonic acid containing 715 ppm by weight of 2-methyl-2-propenyl-1-sulfonic acid. Proportion D is 60%.

### Example 9 (Invention)

The same preparation as in Example 3 is performed with the only difference being that 30% of the 2-acrylamido-2-methylpropane sulfonic acid, or 94.86 g, are substituted with 2-acrylamido-2-methylpropane sulfonic acid containing 1210 ppm IBSA. Proportion D is 30%.

The weight-average molecular weight of the polymers of Examples 1 to 9 is measured according to the method described above, and the results are presented in Table 1.

TABLE 1
Weight-average molecular weight of polymers 1 to
9, R ratio and D proportion of polymers 4 to 9.

| | Weight-average | | (IBSA) | | |
|---|---|---|---|---|---|
| | molecular weight | | (ppm with | | |
| | (in millions | | respect to | | Proportion |
| Example | of g/mol) | C | quantity A) | R | D |
| 1 | 1.12 | — | — | — | |
| 2 | 6.78 | — | — | — | |
| 3 | 11.23 | — | — | — | |
| 4 | 1.10 | 0.60 | 1042 | 232.6 | 53% |
| 5 | 6.81 | 0.60 | 715 | 79.8 | 55% |
| 6 | 11.22 | 0.80 | 1210 | 9.5 | 23% |
| 7 | 1.05 | 0.60 | 1042 | 243.7 | 58% |
| 8 | 6.72 | 0.60 | 715 | 80.9 | 60% |
| 9 | 11.08 | 0.80 | 1210 | 9.6 | 30% |

Thus, substitution of some of the ATBS of good purity (<200 ppm of IBSA) with ATBS of poor purity (250-20,000 ppm of IBSA) does not significantly impact the molecular weight of the polymers.

### Example 10—Application Test 1

Polymers 2, 3, 5, 6, 8 and 9 are dissolved in tap water in order to obtain aqueous solutions having a concentration of 0.1% by weight of the polymer relative to the total weight of the solution. The solutions are stirred mechanically at 200 rpm until the complete solubilization of the polymers and clear and homogeneous solutions are obtained.

A series of flocculation tests are carried out on an aqueous effluent containing 30 g/L of Kaolin, 1 g/L of calcium chloride and 100 g/L of crushed ore.

The tests are carried out in Manual Jar Test according to the following protocol:
tubes are filled with the effluent;
different dosages of a polymeric solution are injected;
5 reversals of the Jar Test are carried out for incorporating the polymeric aqueous solution in the effluent suspension.

The results presented in Table 2 summarize the sedimentation rate according to the dosage of polymer used in relation to the quantity of effluent.

TABLE 2
Results of flocculation tests

Polymer Dosage (g/ton)

| | | | |
|---|---|---|---|
| 4 | 8 | 12 | 16 |

| | |
|---|---|
| Example | Sedimentation Rate (m/h) |

| | | | | |
|---|---|---|---|---|
| 2 | 10 | 22 | 41 | 65 |
| 3 | 12 | 24 | 41 | 63 |
| 5 | 11 | 23 | 42 | 64 |
| 6 | 10 | 22 | 43 | 64 |
| 8 | 10 | 22 | 42 | 62 |
| 9 | 9 | 21 | 41 | 62 |

Thus, substitution of one part of the ATBS of good purity (<250 ppm of IBSA) with ATBS of poor purity (250-20,000 ppm of IBSA) does not significantly impact the molecular weight of the polymers.

### Example 11: Application Test 2

Solutions of polymers 1, 4 and 7 are prepared at an active concentration of 1,000 ppm by weight in a brine containing water, 30,000 ppm by weight of NaCl and 3,000 ppm by weight of CaCl₂·2H₂O. The polymers are tested in an enhanced oil recovery application by injection of polymer solutions. The filtration quotient (FR) is measured on filters having a pore size of 1.2 μm representative of low permeability deposits.

The term filtration quotient (or filter ratio denoted “FR”) is used in this document to designate a test used to determine the performance of the polymer solution under conditions approaching the permeability of the deposit consisting in measuring the time taken by given volumes/concentrations of solution to pass through a filter. The FR generally compares the filterability of the polymer solution for two consecutive equivalent volumes, which indicates the tendency of the solution to clog the filter. Lower FRs indicate better performance.

The test used to determine the FR consists of measuring the times it takes for given volumes of solution containing 1000 active ppm of polymer to flow through a filter. The solution is contained in a pressurized cell at two bars of pressure and the filter is 47 mm in diameter and of defined pore size. Generally, FR is measured with filters having a pore size of 1.2 μm, 3 μm, 5 μm or 10 μm. In the example, the pore size is 1.2 μm.

The times required to obtain 100 mL (t_{100mL}); 200 mL (t_{200mL}) and 300 mL (t_{300mL}) of filtrate are therefore measured. The filtration quotient FR is defined by:

$$FR = \frac{{t}_{300 mL} - {t}_{200 mL}}{{t}_{200 mL} - {t}_{`100 mL}}$$

Times are measured to the nearest 0.1 second.

The FR thus represents the capacity of the polymer solution to clog the filter for two equivalent consecutive volumes.

The results are shown in Table 3 below.

TABLE 3
FR Results

| Example | FR |
|---|---|
| 1 | 1.16 |
| 4 | 1.16 |
| 7 | 1.17 |

Thus, substituting part of good purity ATBS (<300 ppm IBSA, or even <250 or <200 ppm IBSA) with ATBS of poor purity (250-20,0000 ppm IBSA or 300-20,0000) does not significantly impact polymer performance.

1. A polymer obtained at least from a quantity A of 2-acrylamido-2-methylpropane sulfonic acid, in acid and/or salified form, characterized in that quantity A of 2-acrylamido-2-methylpropane acid sulfonic acid contains 250 to 20,000 ppm by weight of 2-methyl-2-propenyl-sulfonic acid, in acid and/or salified form.

2. The polymer according to claim 1, characterized in that quantity A of 2-acrylamido-2-methylpropane sulfonic acid, in acid and/or salified form, contains 300 to 20,000 ppm of 2-methyl-2-propenyl-sulfonic, in acid and/or salified form.

3. The polymer according to claim 1, characterized in that quantity A of 2-acrylamido-2-methylpropanesulfonic acid, in acid and/or salified form, contains 300 to 10,000 ppm of 2-methyl-2-propenylsulfonic acid, in acid and/or salified form.

4. The polymer according to claim 1, characterized in that the polymer is further obtained from a quantity B of 2-acrylamido-2-methylpropane sulfonic acid, in acid and/or salified form, containing strictly less than 200 ppm by weight of 2-methyl-2-propenyl sulfonic acid, in acid and/or salified form.

5. The polymer according to claim 1, characterized in that the polymer is a water-soluble polymer or a superabsorbent polymer.

6. The polymer according to claim 1, characterized in that the polymer is water-soluble and has a weight-average molecular weight greater than 100,000 g/mol and less than or equal to 40 million g/mol.

7. The polymer according to claim 1, characterized in that the polymer is a homopolymer of 2-acrylamido-2-methylpropane sulfonic acid, in acid and/or salified form, or a copolymer of 2-acrylamido-2-methylpropane sulfonic acid, in acid and/or salified form, further comprising at least one monomer chosen from the group comprising nonionic monomers, anionic monomers, cationic monomers, zwitterionic monomers, and mixtures thereof.

8. The polymer according to claim 1, characterized in that the polymer is obtained at least from 2-acrylamido-2-methylpropane sulfonic acid, in acid and/or salified form, the total quantity of which contains strictly less than 20,000 ppm by weight of 2-methyl-2-propenyl sulfonic acid, in acid and/or salified form.

9. The polymer according to claim 1, characterized in that the polymer is water-soluble and in that the proportion D of quantity A relative to the total quantity of 2-acrylamido-2-methylpropane sulfonic acid, in acid and/or salified form, is determined from the ratio R defined according to the following equation (1):
$$R = \frac{{10}^{14}}{{[ IBSA ]}^{2} * {C}^{2} Mw} \qquad ( 1 )$$
wherein,
[IBSA] is the concentration in ppm by weight in quantity A of 2-methyl-2-propenyl-1-sulfonic acid;
Mw is the weight-average molecular weight of the polymer expressed in g/mol;
C is a coefficient equal to 0.2 when Mw is less than or equal to 1 million, equal to 0.6 when Mw is strictly greater than 1 million and less than or equal to 10 million, and equal to 0.8 when Mw is strictly greater than 10 million;
when R is strictly greater than 100, D is between 1 and 100%;
when R is strictly greater than 50 and less than or equal to 100, D is between 1 and 90%;
when R is strictly greater than 10 and less than or equal to 50, D is between 1 and 80%;
when R is strictly greater than 5 and less than or equal to 10, D is between 1 and 60%;
when R is strictly greater than 1 and less than or equal to 5, D is between 1 and 50%;
when R is strictly greater than 0.1 and less than or equal to 1, D is between 1 and 30%;
when R is strictly greater than 0.01 and less than or equal to 0.1, D is between 1 and 10%;
when R is less than or equal to 0.01, D is between 1 and 5%.

10. The polymer according to claim 9, characterized in that
when R is strictly greater than 100, D is between 25 and 95%;
when R is strictly greater than 50 and less than or equal to 100, D is between 25 and 85%;
when R is strictly greater than 10 and less than or equal to 50, D is between 25 and 75%;
when R is strictly greater than 5 and less than or equal to 10, D is between 2 and 50%;
when R is strictly greater than 1 and less than or equal to 5, D is between 2 and 40%;
when R is strictly greater than 0.1 and less than or equal to 1, D is between 2 and 25%;
when R is strictly greater than 0.01 and less than or equal to 0.1, D is between 1 and 8%;
when R is less than or equal to 0.01, D is between 1 and 4%.

11. The polymer according to claim 9, characterized in that
when R is strictly greater than 100, D is between 50 and 90%;
when R is strictly greater than 50 and less than or equal to 100, D is between 50 and 80%;
when R is strictly greater than 10 and less than or equal to 50, D is between 50 and 70%;
when R is strictly greater than 5 and less than or equal to 10, D is between 5 and 40%;
when R is strictly greater than 1 and less than or equal to 5, D is between 4 and 30%;
when R is strictly greater than 0.1 and less than or equal to 1, D is between 3 and 20%;
when R is strictly greater than 0.01 and less than or equal to 0.1, D is between 1 and 6%;
when R is less than or equal to 0.01, D is between 1 and 3%.

12. The polymer according to claim 1, characterized in that quantity A of 2-acrylamido-2-methylpropane sulfonic acid, in acid and/or salified form, additionally contains between 300 and 10,000 ppm by weight 2-methylidene-1,3-propylenedisulfonic acid.

13. Use of the polymer of claim 1 in a field selected from oil and gas recovery, water treatment, sludge treatment, papermaking, construction, mining, cosmetic formulation, detergent formulation, textile manufacturing, and agriculture.

14. Use of the polymer according to claim 1 as a flocculant, viscosity-reducing agent, thickening agent, absorbing agent, friction-reducing agent or plasticizer or superplasticizer.

15. The polymer according to claim 3, characterized in that the polymer is further obtained from a quantity B of 2-acrylamido-2-methylpropane sulfonic acid, in acid and/or salified form, containing strictly less than 200 ppm by weight of 2-methyl-2-propenyl sulfonic acid, in acid and/or salified form.

16. The polymer according to claim 15, characterized in that the polymer is water-soluble and has a weight-average molecular weight greater than 100,000 g/mol and less than or equal to 40 million g/mol.

17. The polymer according to claim 16, characterized in that the polymer is a homopolymer of 2-acrylamido-2-methylpropane sulfonic acid, in acid and/or salified form, or a copolymer of 2-acrylamido-2-methylpropane sulfonic acid, in acid and/or salified form, further comprising at least one monomer chosen from the group comprising nonionic monomers, anionic monomers, cationic monomers, zwitterionic monomers, and mixtures thereof.

18. The polymer according to claim 17, characterized in that the polymer is obtained at least from 2-acrylamido-2-methylpropane sulfonic acid, in acid and/or salified form, the total quantity of which contains strictly less than 20,000 ppm by weight of 2-methyl-2-propenyl sulfonic acid, in acid and/or salified form.

19. The polymer according to claim 18, characterized in that the proportion D of quantity A relative to the total quantity of 2-acrylamido-2-methylpropane sulfonic acid, in acid and/or salified form, is determined from the ratio R defined according to the following equation (1):
$$R = \frac{{10}^{14}}{{[ IBSA ]}^{2} * {C}^{2} Mw} \qquad ( 1 )$$
wherein,
[IBSA] is the concentration in ppm by weight in quantity A of 2-methyl-2-propenyl-1-sulfonic acid;
Mw is the weight-average molecular weight of the polymer expressed in g/mol;
C is a coefficient equal to 0.2 when Mw is less than or equal to 1 million, equal to 0.6 when Mw is strictly greater than 1 million and less than or equal to 10 million, and equal to 0.8 when Mw is strictly greater than 10 million;
when R is strictly greater than 100, D is between 1 and 100%;
when R is strictly greater than 50 and less than or equal to 100, D is between 1 and 90%;
when R is strictly greater than 10 and less than or equal to 50, D is between 1 and 80%;
when R is strictly greater than 5 and less than or equal to 10, D is between 1 and 60%;
when R is strictly greater than 1 and less than or equal to 5, D is between 1 and 50%;
when R is strictly greater than 0.1 and less than or equal to 1, D is between 1 and 30%;
when R is strictly greater than 0.01 and less than or equal to 0.1, D is between 1 and 10%;
when R is less than or equal to 0.01, D is between 1 and 5%.

20. The polymer according to claim 19, characterized in that quantity A of 2-acrylamido-2-methylpropane sulfonic acid, in acid and/or salified form, additionally contains between 300 and 10,000 ppm by weight 2-methylidene-1,3-propylenedisulfonic acid; and
when R is strictly greater than 100, D is between 50 and 90%;
when R is strictly greater than 50 and less than or equal to 100, D is between 50 and 80%;
when R is strictly greater than 10 and less than or equal to 50, D is between 50 and 70%;
when R is strictly greater than 5 and less than or equal to 10, D is between 5 and 40%;
when R is strictly greater than 1 and less than or equal to 5, D is between 4 and 30%;
when R is strictly greater than 0.1 and less than or equal to 1, D is between 3 and 20%;
when R is strictly greater than 0.01 and less than or equal to 0.1, D is between 1 and 6%;
when R is less than or equal to 0.01, D is between 1 and 3%.
